(19) United States
(12) Patent Application Publication
(10) Pub. No.: US 2023/0201207 A1
(43) Pub. Date: Jun. 29, 2023

(54) COMBINATION CANCER THERAPY USING N2-QUINOLINE OR ISOQUINOLINE SUBSTITUTED PURINE DERIVATIVES

(71) Applicant: SHANGHAI HUAYU BIOTECHNOLOGY CO., LTD., Shanghai (CN)

(72) Inventor: Zhanggui WU, Brookline, MA (US)

(21) Appl. No.: 17/996,444

(22) PCT Filed: May 3, 2021
(86) PCT No.: PCT/US21/30402

§ 371 Date: Oct. 18, 2022

Related U.S. Application Data
(60) Provisional application No. 63/021,206, filed on May 7, 2020.

(51) Int. Cl. A61K 31/52; A61P 35/02; C07K 16/28

(52) U.S. Cl. CPC A61K 31/52; A61P 35/02; C07K 16/2896

(57) ABSTRACT

The present invention relates to the use of N2-quinoline or isoquinoline substituted purine derivatives in cancer treatment in combination with an immunotherapeutic agent or a therapeutic agent targeting a cancer-promoting/sustaining molecule.

### RELATED APPLICATIONS AND INCORPORATION BY REFERENCE

This application claims priority to U.S. Provisional Patent Application No. 63/021,206 filed May 7, 2020.

All documents cited or referenced herein (including without limitation all literature documents, patents, published patent applications cited herein) (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference. Any Genbank sequences mentioned in this disclosure are incorporated by reference with the Genbank sequence to be that of the earliest effective filing date of this disclosure.

### TECHNICAL FIELD

The present disclosure relates to the use of N²-quinoline or isoquinoline substituted purine derivatives in combination with an immunotherapeutic agent or a therapeutic agent targeting a cancer-promoting/sustaining molecule in cancer treatment.

### BACKGROUND OF THE INVENTION

Cancer is a group of diseases involving abnormal cell growth, with the potential to spread to other parts of body, causing about 600,000 people die in the United States in 2019.

The current options for cancer treatment include surgery, radiation therapy, chemotherapy, hormone therapy, and immunotherapy. The traditional radiation therapy and chemotherapy were and still are first-line treatments for various types of cancers due to efficient destruction of cancer cells. However, these two commonly adopted therapies are toxic to patients as they usually non-selectively destroy healthy cells and cause depression of the immune system. Cancer immunotherapy involves activation of the immune system and amplification of immune responses, and was voted “breakthrough of the year” by Science in 2013. It offers a lesser high-grade toxicity compared with other stand therapies, and enjoyed an unparalleled success over its peers, but a subset of patients does not respond to it as a monotherapy probably because cancer cells develop several mechanisms to evade immune surveillance and induce immune tolerance. Targeted therapy is a medical treatment that partly overlaps the chemotherapy and immunotherapy where chemotherapeutic agents or biopharmaceuticals interfere with specific targeted molecules needed for carcinogenesis and/or tumor growth, and is sometimes less harmful to healthy cells than the traditional cytotoxic chemotherapy.

Combination therapy has emerged as a promising new cancer treatment strategy, as the combination of two or more therapeutic treatments may target more than one cancer-inducing or sustaining pathways so as to increase the chance of killing cancer cells, minimize drug resistance and lower single drug dose. However, not all therapies can be combined and even few combination treatments work in a synergistic manner, as one therapeutic agent may change a secondary agent's conformation and thus disable its anti-tumor activity. Alternatively, one therapeutic agent may inhibit the metabolism of a secondary agent in human body, leading to the buildup of toxicity. For example, in a pooled analysis of 14 phase I-III studies, while 64% of patients receiving various doses of Ipilimumab (anti-CTLA-4) experienced immune-related adverse effects, patients receiving dual therapy with anti-PD-1 mAb plus Ipilimumab had a 93% incidence of adverse events (Wolchok, J. D., et al., Nivolumab plus ipilimumab in advanced melanoma. N. Engl. J. Med., 2013. 369: 122-33). The combination of panobinostat and carfilzomib caused treatment-related heart failure (2%), and treatment-related death rose by 2% in patients with relapsed/refractory multiple myeloma (Berdeja J G et al., (2015) Haematologica 100(5):670-676). Full investigation is thus needed on the interaction between two or more anti-tumor agents in a combination regimen before coming to a conclusion that whether the combination regimen is proper or not, or whether a maximum efficacy may be achieved with minimal toxicity.

The N²-quinoline or isoquinoline substituted purine derivatives disclosed in US2006/0293274A have been found to inhibit cancer cell growth in vitro and in viva but may at the same time adversely affect cellular processes in healthy cells and suppress the immune system to some extent.

Citation or identification of any document in this application is not an admission that such document is available as prior art to the present invention.

### SUMMARY OF THE INVENTION

The present inventor has surprisingly found that the purine derivatives of US2006/0293274A1 work synergistically with an anti-PD-1 antibody to provide an enhanced anti-tumor effect which does not elevate toxicity. Other immunotherapeutic or therapeutic agents that target a cancer promoting/sustaining molecule, such as but not limited to a PD-1/PD-L1 inhibitor, a CTLA 4 inhibitor or an ErbB inhibitor are contemplated for the present invention. Therefore, this combination of compounds with an optional immunotherapeutic agent or a therapeutic agent are useful for cancer treatment.

Therefore, in one aspect, the present disclosure relates to a method for treating cancer in a subject in need thereof, which may comprise administering to the subject a therapeutically effective amount of a compound of formula (I), or a pharmaceutically acceptable salt or solvent thereof, in combination with an immunotherapeutic agent or a therapeutic agent targeting a cancer-promoting/sustaining molecule,

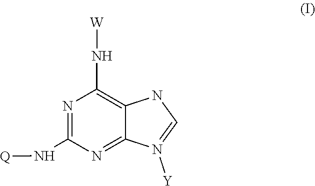

wherein W is hydrogen, an optionally substituted C₁₋₆ alkyl, an optionally substituted C₃₋₆ cycloalkyl, or an optionally substituted C₁₋₆ haloalkyl,
Y is hydrogen, or a saccharide, and
Q is hydrogen, or one of the following:

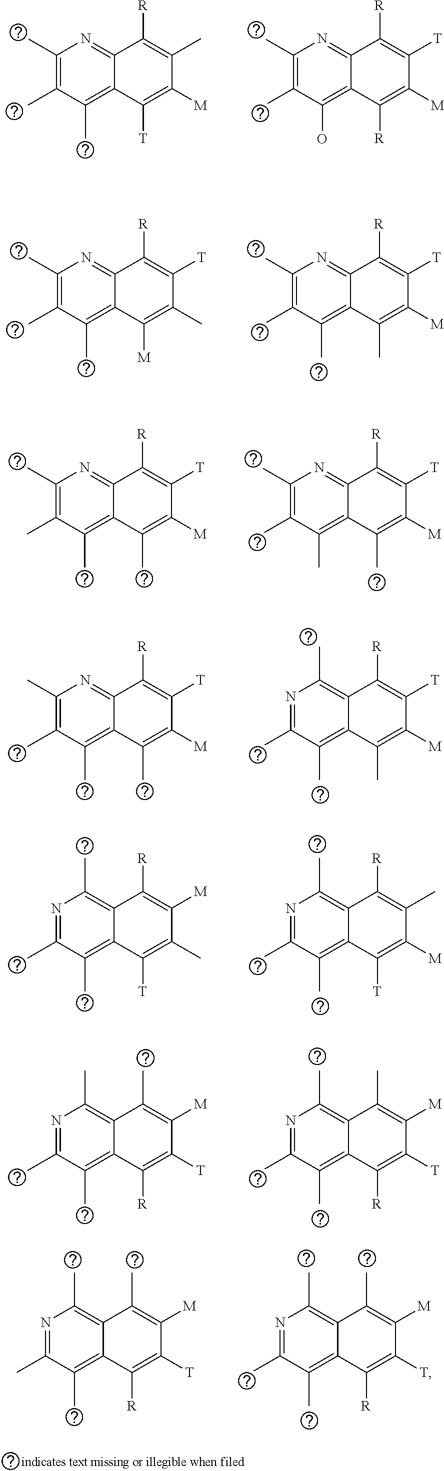

wherein B, E, G, R, T and M are independently hydrogen, an C₁₋₆ alkyl, an C₃₋₆ cycloalkyl, a halogen, a cyano, or an amino group.

W in formula (I) is preferably one of the following:

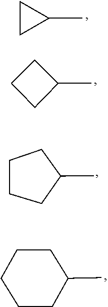

Q in formula (I) is preferably one of the following:

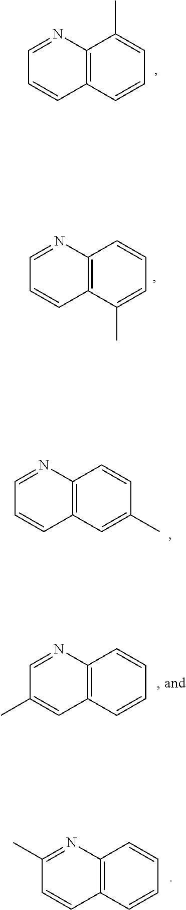

When Y in formula I is a saccharide, it is pharmaceutically acceptable and preferably one of the following:

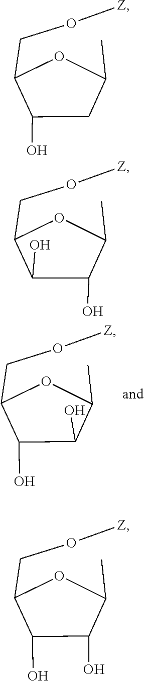

wherein Z is hydrogen or one of the following:

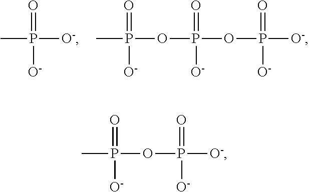

In one embodiment, W is preferably

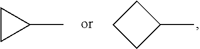

and Q is one of the following:

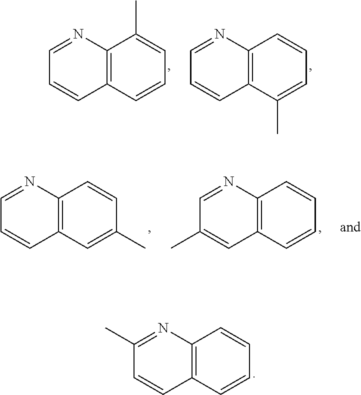

Q is preferably

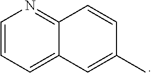

The compound of formula (I) may be selected from the group consisting of:

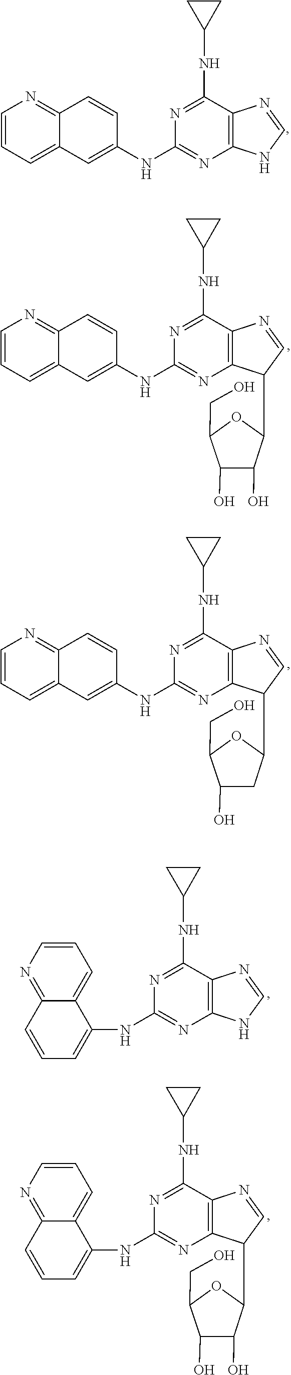

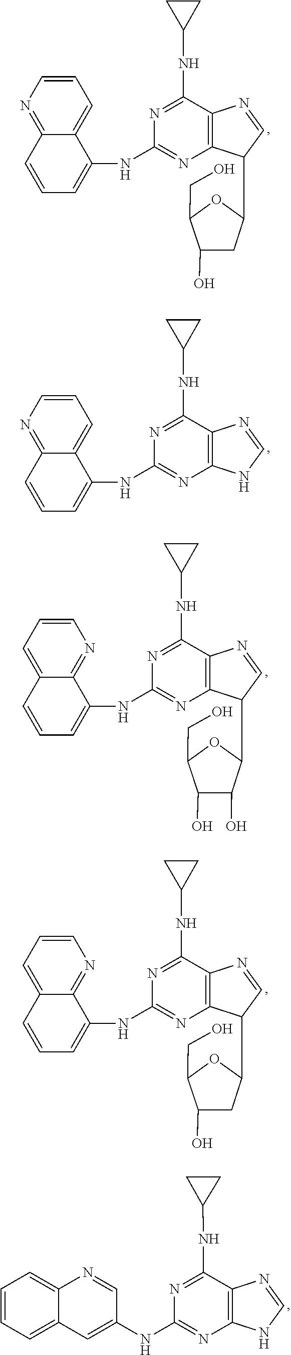

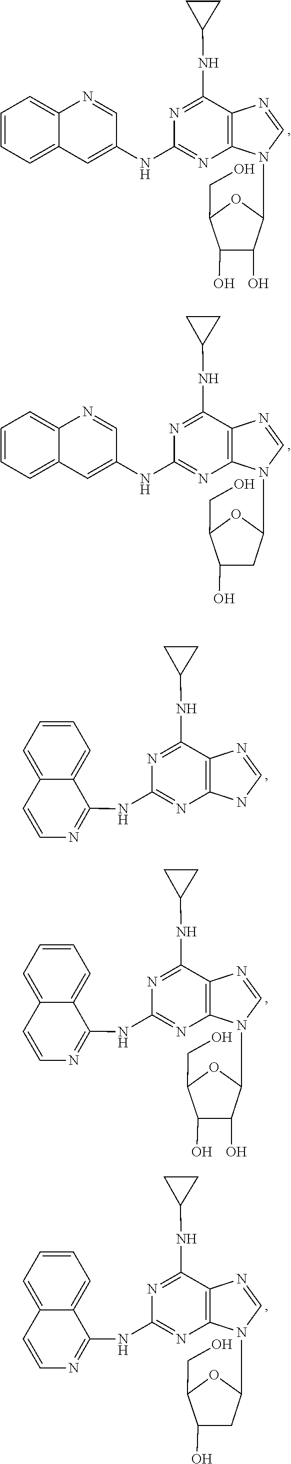

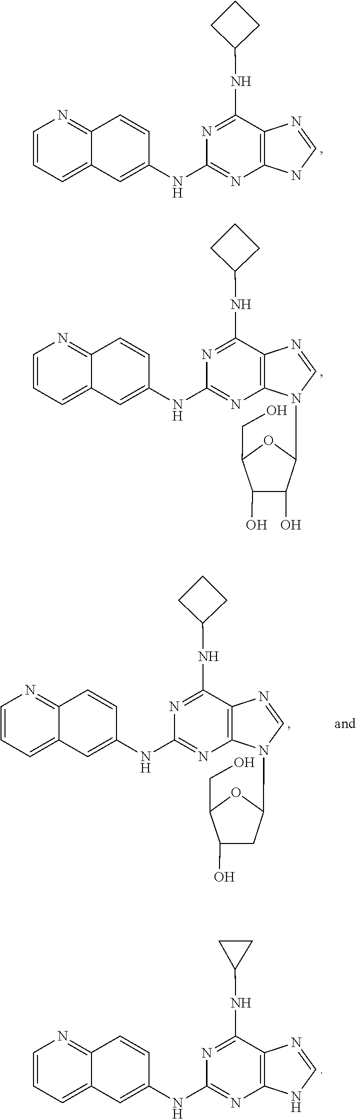

A preferable compound is

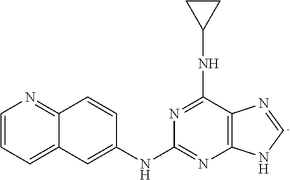 [SMILES: c1cnc2ccc(Nc3nc(NC4CC4)c4nc[nH]c4n3)cc2c1]

with an extremely high activity, also referred to as Compound A hereinafter.

The immunotherapeutic agent or the therapeutic agent targeting a cancer-promoting/sustaining molecule may be an inhibitor of PD-1, PD-L1, CTLA-4, HER-2, CD20, CD33, or CD52. In embodiments, the immunotherapeutic agent or the therapeutic agent targeting a cancer-promoting/sustaining molecule may be an antibody targeting PD-1, PD-L1, CTLA4, HER-2, CD20, CD33, and/or CD52, and an antibody-drug conjugate (ADC) or a CAR-T cell targeting PD-L1, HER-2, CD20, CD33, and/or CD52.

In one embodiment, the immunotherapeutic agent or the therapeutic agent targeting a cancer-promoting/sustaining molecule may be a PD-1 inhibitor, such as an anti-PD-1 antibody; or a PD-L1 inhibitor such as an anti-PD-L1 antibody. The anti-PD-1 antibody can be Nivolumab or Pembrolizumab. The anti-PD-L1 antibody may be Atezolizumab, Druvalumab or Avelumab.

The CTLA-4 inhibitor may be an anti-CTLA-4 antibody, such as Ipilimumab. The anti-HER2 antibody may be Trastuzumab or Pertuzumab. The anti-CD-20 antibody may be Rituximab, Ibritumomab, Tiuxetan, Tositumomab, Ofatumumab, Ocrelizumab, Veituzumab or Obinutuzumab. The anti-CD-33 antibody may be Gemtuzumab. The anti-CD-52 antibody may be Alemtuzumab.

Other inhibitors of PD-1, PD-L1, CTLA-4, HER-2, CD20, CD33, or CD52 include, but are not limited to, antisense or RNAi, or other compounds that inhibit the expression of PD-1, PD-L1, CTLA-4, HER-2, CD20, CD33, or CD52.

In one embodiment, the cancer may be a solid cancer selected from the group consisting of lung, prostate, ovarian, brain, breast, skin, bladder, colon, gastrointestinal, head and neck, gastric, pancreas, neurologic, renal, and liver cancer. In one embodiment, the cancer may be colon cancer.

In one embodiment, the cancer may be a hematological cancer selected from the group consisting of lymphocytic leukemia, myeloid leukemia, non-Hodgkin lymphoma, and Hodgkin lymphoma. In one embodiment, the cancer may be myeloid leukemia, especially acute myeloid leukemia (AML).

The racemates, pure stereoisomers, in particular enantiomers or diastereomers, and the mixtures of stereoisomers in any mixing ratio of the compounds described above may be also used in the cancer treatment method of the disclosure.

The present disclosure also relates to the use of the compound of formula (I) in cancer treatment in combination with the immunotherapeutic agent or the therapeutic agent targeting a cancer-promoting/sustaining molecule.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### DETAILED DESCRIPTION OF THE INVENTION

Before particular embodiments of the present disclosure are disclosed and described, it is to be understood that this disclosure is not limited to the particular process and materials disclosed herein as such may vary to some degree. It is also to be understood that the terminology used herein is used for the purpose of describing particular embodiments only and is not intended to be limiting, as the scope of the present disclosure will be defined only by the appended claims and equivalents thereof.

The singular forms “a,” “an,” and “the” include plural referents unless the context clearly dictates otherwise.

Unless otherwise indicated, any heteroatom with unsatisfied valences is assumed to have hydrogen atoms sufficient to satisfy the valences.

Throughout the specification and the appended claims, a given chemical formula or name shall encompass all stereo and optical isomers and racemates thereof where such isomers exist. Unless otherwise indicated, all chiral (enantiomeric and diastereomeric) and racemic forms are within the scope of the disclosure. Many geometric isomers of C═C double bonds, C═N double bonds, ring systems, and the like can also be present in the compounds, and all such stable isomers are contemplated in the present disclosure. Cis- and trans- (or E- and Z-) geometric isomers of the compounds of the present disclosure are described and may be isolated as a mixture of isomers or as separated isomeric forms. The present compounds can be isolated in optically active or racemic forms. Optically active forms may be prepared by resolution of racemic forms or by synthesis from optically active starting materials. All processes used to prepare compounds of the present disclosure and intermediates made therein are considered to be part of the present disclosure. When enantiomeric or diastereomeric products are prepared, they may be separated by conventional methods, for example, by chromatography or fractional crystallization. Depending on the process conditions the end products of the present disclosure are obtained either in free (neutral) or salt form. Both the free form and the salts of these end products are within the scope of the disclosure. If so desired, one form of a compound may be converted into another form. A free base or acid may be converted into a salt; a salt may be converted into the free compound or another salt; a mixture of isomeric compounds of the present disclosure may be separated into the individual isomers. Compounds of the present disclosure, free form and salts thereof, may exist in multiple tautomeric forms, in which hydrogen atoms are transposed to other parts of the molecules and the chemical bonds between the atoms of the molecules are consequently rearranged. It should be understood that all tautomeric forms, insofar as they may exist, are included within the disclosure.

As referred to herein, the term “substituted” means that at least one hydrogen atom is replaced with a non-hydrogen group, provided that normal valencies are maintained and that the substitution results in a stable compound.

When a substituent is noted as “optionally substituted”, the substituents are selected from, for example, substituents such as alkyl, cycloalkyl, aryl, heterocyclo, halo, hydroxy, alkoxy, oxo, alkanoyl, aryloxy, alkanoyloxy, amino, alkylamino, arylamino, arylalkylamino, disubstituted amines in which the 2 amino substituents are selected from alkyl, aryl or arylalkyl; alkanoylamino, aroylamino, aralkanoylamino, substituted alkanoylamino, substituted arylamino, substituted aralkanoylamino, thiol, alkylthio, arylthio, arylalkylthio, alkylthiono, arylthiono, arylalkylthiono, alkylsulfonyl, arylsulfonyl, arylalkylsulfonyl, sulfonamido, e.g. —SO₂NH₂, substituted sulfonamido, nitro, cyano, carboxy, carbamyl, e.g. —CONH₂, substituted carbamyl e.g. —CONHalkyl, —CONHaryl, —CONHarylalkyl or cases where there are two substituents on the nitrogen selected from alkyl, aryl or arylalkyl; alkoxycarbonyl, aryl, substituted aryl, guanidino, heterocyclyl, e.g., indolyl, imidazolyl, furyl, thienyl, thiazolyl, pyrrolidyl, pyridyl, pyrimidyl, pyrrolidinyl, piperidinyl, morpholinyl, piperazinyl, homopiperazinyl and the like, and substituted heterocyclyl, unless otherwise defined.

As used herein, the term “alkyl” or “alkylene” is intended to include both branched and straight-chain saturated aliphatic hydrocarbon groups having the specified number of carbon atoms. For example, “C₁-C₆ alkyl” denotes alkyl having 1 to 6 carbon atoms. Exemplary alkyl groups include, but are not limited to, methyl (Me), ethyl (Et), propyl (e.g., n-propyl and isopropyl), butyl (e.g., n-butyl, isobutyl, t-butyl), and pentyl (e.g., n-pentyl, isopentyl, neopentyl).

The term “alkenyl” denotes a straight- or branch-chained hydrocarbon radical containing one or more double bonds and typically from 2 to 20 carbon atoms in length. For example, “C₂-C₈ alkenyl” contains from two to eight carbon atoms. Alkenyl groups include, but are not limited to, for example, ethenyl, propenyl, butenyl, 1-methyl-2-buten-1-yl, heptenyl, octenyl and the like.

The term “alkynyl” denotes a straight- or branch-chained hydrocarbon radical containing one or more triple bonds and typically from 2 to 20 carbon atoms in length. For example, “C₂-C₈ alkenyl” contains from two to eight carbon atoms. Representative alkynyl groups include, but are not limited to, for example, ethynyl, 1-propynyl, 1-butynyl, heptynyl, octynyl and the like.

The term “alkoxy” or “alkyloxy” refers to an —O-alkyl group. “C₁-C₆ alkoxy” (or alkyloxy), is intended to include C₁, C₂, C₃, C₄, C₅, and C alkoxy groups. Exemplary alkoxy groups include, but are not limited to, methoxy, ethoxy, propoxy (e.g., n-propoxy and isopropoxy), and t-butoxy. Similarly, “alkylthio” or “thioalkoxy” represents an alkyl group as defined above with the indicated number of carbon atoms attached through a sulphur bridge; for example methyl-S— and ethyl-S—.

The term “aryl”, either alone or as part of a larger moiety such as “aralkyl”, “aralkoxy”, or aryloxyalkyl”, refers to monocyclic, bicyclic and tricyclic ring systems having a total of five to 15 ring members, wherein at least one ring in the system is aromatic and wherein each ring in the system contains three to seven ring members. In certain embodiments of the disclosure, “aryl” refers to an aromatic ring system which includes, but not limited to phenyl, biphenyl, indanyl, 1-naphthyl, 2-naphthyl and terahydronaphthyl. The term “aralkyl” or “arylalkyl” refers to an alkyl residue attached to an aryl ring. Non-limiting examples include benzyl, phenethyl and the like. The fused aryls may be connected to another group either at a suitable position on the cycloalkyl ring or the aromatic ring. For example:

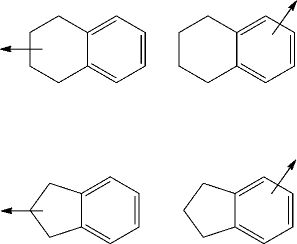

Arrowed lines drawn from the ring system indicate that the bond may be attached to any of the suitable ring atoms.

The term “cycloalkyl” refers to cyclized alkyl groups. C₃-C₆ cycloalkyl is intended to include C₃, C₄, C₅, and C₆ cycloalkyl groups. Exemplary cycloalkyl groups include, but are not limited to, cyclopropyl, cyclobutyl, cyclopentyl, cyclohexyl, and norbornyl. Branched cycloalkyl groups such as 1-methylcyclopropyl and 2-methylcyclopropyl are included in the definition of “cycloalkyl”. The term “cycloalkenyl” refers to cyclized alkenyl groups. C₄₋₆ cycloalkenyl is intended to include C₄, C₅, and C₆ cycloalkenyl groups. Exemplary cycloalkenyl groups include, but are not limited to, cyclobutenyl, cyclopentenyl, and cyclohexenyl.

As used herein, the term “heterocycle,” “heterocyclyl,” or “heterocyclic group” is intended to mean a stable 4-, 5-, or 6-membered monocyclic that is saturated, partially unsaturated, or fully unsaturated, and that contains carbon atoms and 1, 2, 3 or 4 nitrogen, oxygen or other non-carbon atoms.

In cases wherein there are nitrogen atoms (e.g., amines) on compounds of the present disclosure, these may be converted to N-oxides by treatment with an oxidizing agent (e.g., mCPBA and/or hydrogen peroxides) to afford other compounds of this disclosure. Thus, shown and claimed nitrogen atoms are considered to cover both the shown nitrogen and its N-oxide (N→0) derivative.

When any variable occurs more than one time in any constituent or formula for a compound, its definition at each occurrence is independent of its definition at every other occurrence. Thus, for example, if a group is shown to be substituted with 0-3 R, then said group may optionally be substituted with up to three R groups, and at each occurrence R is selected independently from the definition of R. Also, combinations of substituents and/or variables are permissible only if such combinations result in stable compounds.

When a bond to a substituent is shown to cross a bond connecting two atoms in a ring, then such substituent may be bonded to any atom on the ring. When a substituent is listed without indicating the atom in which such substituent is bonded to the rest of the compound of a given formula, then such substituent may be bonded via any atom in such substituent. Combinations of substituents and/or variables are permissible only if such combinations result in stable compounds.

The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, and/or other problem or complication, commensurate with a reasonable benefit/risk ratio. As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like. A “pharmaceutically acceptable solvate” refers to a multicomponent crystalline solid molecular adduct containing the hose molecule (e.g., the compound of formula (I)) and guest solvent molecule(s) incorporated in the crystal lattice structure. When the solvent is water, the solvate is called hydrate. A “pharmaceutically acceptable carrier” refers to media generally accepted in the art for the delivery of biologically active agents to animals, in particular, mammals, including, i.e., adjuvant, excipient or vehicle, such as diluents, preserving agents, fillers, flow regulating agents, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington: The Science and Practice of Pharmacy, 22nd Edition, Allen, L. V. Jr., Ed.; Pharmaceutical Press, London, UK (2012), the disclosure of which is hereby incorporated by reference.

As used herein, the term “effective amount” means that amount of a drug or pharmaceutical agent, i.e., a compound of the disclosure, that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. An effective amount can be administered in one or more administrations, applications or dosages and is not intended to be limited to a particular formulation or administration route. The term also includes within its scope amounts effective to enhance normal physiological function

As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “parenteral” includes subcutaneous, intradermal, intravenous, intramuscular, intraarticular, intraarterial, synovial, intrasternal, intracranial, intramuscular or infusion.

The term “subject” includes any human or nonhuman animal. The term “nonhuman animal” includes all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses.

The term “IC₅₀” or “half maximal inhibitory concentration” refers to the concentration of an inhibitor where the response is reduced by half.

The term “lethal dose” or “LD”, in toxicology, is an indication of the lethal toxicity of a given substance such as the compound of formula (I) in the present disclosure. The median lethal dose, abbreviated as LD₅₀, of the compound refers to the dose required to kill half the members of a tested population after a specified test duration. The term “median toxic dose” or “TD₅₀” of a drug refers to a dose at which toxicity occurs in 50% of cases. The term “efficacious dose” or “ED” refers to a dose of a drug such as the compound of formula (I) in the present disclosure that produces a biological response. The median efficacious dose, abbreviated as ED₅₀, is a dose that produces a quantal effect in 50% of the population that takes the drug. The therapeutic index or therapeutic ratio can be calculated as the ratio of TD50 to ED50, or LD50 to ED50, which is a quantitative measurement of the relative safety of a drug.

The compounds of formula (I) are disclosed in US2006/0293274A1, having low toxicity and high anti-tumor activity, as well as the synthesis scheme and the function tests. An exemplary compound is

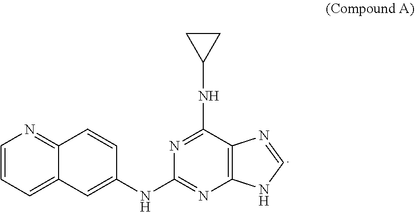 [SMILES: c1cnc2ccc(Nc3nc(NC4CC4)c4nc[nH]c4n3)cc2c1]

The inventor of the disclosure has found that these compounds are capable of inhibiting the growth of many types of cancers in vitro, such as but not limited to, breast cancer, lymphoma, leukemia, osteosarcoma, ovarian cancer, liver cancer, prostate cancer, pancreatic cancer, bladder cancer, prostate cancer, stomach cancer, lung cancer, colon cancer, nasopharyngeal carcinoma, and kidney cancer. Their in vivo anti-tumor activity has also been confirmed in mice bearing Lewis lung carcinomas and H-22 liver cancer. The inventor further found that these compounds are particularly effective in the treatment of leukemia as compared to other cancers. Without bound to the theory, the inventor believes that such obviously better inhibitory effect on leukemia is associated with the compounds' inhibitory effect on phosphoinositide 3-kinase (PI3K) δ isoform and FLT3.

In particular, the compounds of formula (I) are capable of inhibiting several kinases' activities, such as PI3K (including α, β, δ and γ isoforms), Protein kinase B (PKB, also known as AKT) and Platelet-derived growth factor receptor (PDGFR). The present inventor tested the inhibitory effect of Compound A on the four PI3K isoforms, using the method as described in CN104513254A, and found that the EC₅₀ with respect to δ isoform inhibition was about 90 nM, which was much lower than those for the other three isoforms at the μM level. The δ and γ isoforms are mainly expressed in leukocytes (Saudemont et al., (2009) Proc. Nat'l. Acad Sci. 2009106: 5795-5800; Ali et al., (2014) Nature 510: 407-411), and thus the compound selectively against the δ isoform may be more active and efficacious in leukemia treatment.

The inventor further tested the inhibitory effect of Compound A on kinase insert domain receptor (KDR, also referred to as VEGFR-2), PDGFR, AKT and fms-like tyrosine kinase 3 (FLT3). The results showed that this compound inhibited the phosphorylation of KDR, PDGFR, AKT and FLT3 with EC₅₀ at about 1.0 μM, 1.0 μM, 0.1 μM and 49.0 nM, suggesting its high inhibitory activity on FLT3. FLT3 is a class III receptor tyrosine kinase and plays an important role in hematopoiesis and lymphocyte development. The abnormal FLT3 activation was found to be closely associated with the development of several tumors such as AML (Griffith et al., (2004) Mol Cell. 13:169-78). Wang et al found over 60% of 82 leukemia cell lines were FLT3 positive (Wang Y et al., (2006) Journal of Experimental Hematology 14(3): 446-449). FLT3-internal tandem duplication (ITD) is the most commonly seen mutation in AML patients, with about 17-34% of AML patients carrying such mutation (Jia H et al., (2018) Medical Laboratory Science and Clinics 19(5): 39) FTL3 inhibitors such as Quizartinib, Sorafenib and Gilteritinib are clinically used in acute myeloblastic leukemia treatment and resulted in good clinical outcomes (Qi L et al., (2014) Chinese Journal of Cancer Biotherapy 21(1): 20-24). Thus, the compounds of the disclosure with high inhibitory effect on FLT3 function may be good candidates for leukemia treatment.

The immunotherapeutic agent herein refers to a therapeutic agent involved in activation of immune system and/or amplification of immune responses. The immunotherapeutic agent includes, but not limited to, a PD-1 inhibitor, a PD-L1 inhibitor, a CTLA-4 inhibitor, and a CD33 inhibitor. The PD-1 inhibitor, PD-L1 inhibitor, CTLA-4 inhibitor, and CD33 inhibitor include, but not limited to, an anti-PD-1 antibody, an anti-PD-L1 antibody, an anti-CTLA-4 antibody, an anti-CD33 antibody, an anti-PD-L1-antibody-drug conjugate, an anti-CD33-antibody-drug conjugate, a CAR-T cell targeting PD-L1, and a CAR-T cell targeting CD33.

The therapeutic agent targeting a cancer-promoting/sustaining molecule herein refers to a therapeutic agent targeting a molecule required for cancer cell growth, and partly overlaps the immunotherapeutic agent. Such an agent includes, but not limited to, a PD-1 inhibitor, a PD-L1 inhibitor, a CTLA-4 inhibitor, a HER-2 inhibitor, a CD20 inhibitor, a CD33 inhibitor, or a CD52 inhibitor. These inhibitors may be an antibody targeting PD-1, PD-L1, CTLA-4, HER-2, CD20, and/or CD33, an antibody-drug conjugate or a CAR-T cell targeting PD-L1, HER-2, CD20, and/or CD33.

Among these targets, PD-1, PD-L1 and CTLA-4 are immune checkpoints.

PD-1 is a protein found on the surface of cells that has a role in regulating the immune system's response to the cells of the human body by down-regulating the immune system and promoting self-tolerance by suppressing T cell inflammatory activity. PD-1 binds two ligands, PD-L1 and PD-L2. The binding of PD-L1 to PD-1 transmits an inhibitory signal that reduces the proliferation of antigen-specific T-cells in lymph nodes, while simultaneously reducing apoptosis in regulatory T cells (anti-inflammatory, suppressive T cells). PD-1 and PD-L1 inhibitors activate the immune system to attack tumors and are used to treat certain types of cancer, and have been one of the most intensively researched cancer therapeutics. However, only less than about 20% of patients respond PD-1/PD-L1 inhibitors.

CTLA-4, also known as cytotoxic T-lymphocyte antigen 4, is a trans-membrane protein found on the surface of T cells, which, when bound to B7 on antigen-presenting cells, prevents T cell activation. This is thought to occur during the immune system's “priming” phase in lymph nodes. The overall effect is immune down-regulation. Thus, CTLA-4 is described as an immune checkpoint “off switch.” Two fully human anti-CTLA-4 monoclonal antibodies have been developed and tested in phase III clinical trials for the treatment of patients with metastatic melanoma: Ipilimumab and Tremelimumab. Ipilimumab has been FDA-approved for the treatment of metastatic melanoma, adjuvant melanoma, and renal cell carcinoma.

The other targets are known for their roles in promoting cancer generation and/or sustaining cancer growth.

The compounds of formula (I) may be used in combination with an immunotherapeutic agent or a cancer-promoting/sustaining molecule targeting therapeutic agent, to gain a better anti-cancer effect and/or a lower toxicity to human body.

The compound of formula (I) and the immunotherapeutic agent or the cancer-promoting/sustaining molecule targeting therapeutic agent may be administered concurrently as a single composition in a pharmaceutically acceptable carrier, or concurrently as separate compositions. They can also be administered sequentially.

The combination therapy of the present disclosure may be used to treat cancer, such as a solid cancer selected from the group consisting of lung, prostate, ovarian, brain, breast, skin, bladder, colon, gastrointestinal, head and neck, gastric, pancreas, neurologic, renal, and liver cancer, or a hematological cancer selected from the group consisting of lymphocytic leukemia, myeloid leukemia, non-Hodgkin lymphoma, and Hodgkin lymphoma.

The combination therapy of the present disclosure may be applied to animals, preferably mammals (e.g., domesticated animals, cats, dogs, mice, rats), and more preferably humans. Any method of administration may be used to deliver the compound of formula I and the immunotherapeutic agent or the cancer-promoting/sustaining molecule targeting therapeutic agent to the subject in need thereof. In certain embodiments, the compound of formula (I) and the immunotherapeutic agent or the cancer-promoting/sustaining molecule targeting therapeutic agent are administered orally. In other embodiments, the compound of formula I and the immunotherapeutic agent or the cancer-promoting/sustaining molecule targeting therapeutic agent are administered parenterally.

One or more additional pharmaceutical agents or treatment methods such as, for example, other chemotherapeutics or other anti-cancer agents, immune enhancers, immunosuppressants, anti-tumor vaccines, and/or cytokine therapy (e.g., IL2 and GM-CSF) may be optionally used in combination with the combination therapy of the disclosure. The additional agents may be combined with the compound of formula (I) and the immunotherapeutic agent or the cancer-promoting/sustaining molecule targeting therapeutic agent in a single dosage form, or these agents may be administered simultaneously or sequentially as separate dosage forms.

The disclosure also provides pharmaceutically acceptable compositions which comprise a therapeutically effective amount of one or more compounds of Formula (I), formulated together with one or more pharmaceutically acceptable carriers (additives) and/or diluents, with or without a therapeutically effective amount of the immunotherapeutic agent or the cancer-promoting/sustaining molecule targeting therapeutic agent mentioned above, formulated together with one or more pharmaceutically acceptable carriers (additives) and/or diluents, and optionally, one or more additional therapeutic agents described above if needed. The pharmaceutical composition of this disclosure as a whole, or each component in the composition, can be administered by any suitable means, for example, orally, as tablets, capsules (each of which includes sustained release or timed release formulations), pills, powders, granules, elixirs, tinctures, suspensions (including nanosuspensions, microsuspensions, spray-dried dispersions), syrups, and emulsions; sublingually; bucally; parenterally, such as by subcutaneous, intravenous, intramuscular, or intrasternal injection, or infusion techniques (e.g., as sterile injectable aqueous or non-aqueous solutions or suspensions); nasally, including administration to the nasal membranes, such as by inhalation spray; topically, such as in the form of a cream or ointment; or rectally such as in the form of suppositories. The pharmaceutical composition of the present disclosure can also be prepared as liposomes and nanoparticles.

The dosage regimen for the pharmaceutical compositions of the disclosure will, of course, vary depending upon known factors, such as the pharmacodynamic characteristics of the particular agents and the mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. By way of general guidance, the daily oral dosage of each active ingredient, when used for the indicated effects, will range between about 0.001 to about 5000 mg per day, preferably between about 0.01 to about 1000 mg per day, and most preferably between about 0.1 to about 250 mg per day. Intravenously, the most preferred doses will range from about 0.01 to about 10 mg/kg/minute during a constant rate infusion. In some embodiments, the compound of the disclosure is administered every day or every other day at a daily dose of 10-500 mg/kg. In one embodiment, the compound is administered every day at a daily dose of 0.1-200 mg/kg, preferably 0.2-100 mg/kg, and most preferably 0.3-50 mg/kg.

The pharmaceutical composition of this disclosure may be administered in a single daily dose, or the total daily dosage may be administered in divided doses of two, three, or four times daily.

The toxicity and efficacy of the pharmaceutical compositions, with or without the immunotherapeutic agent or the cancer-promoting/sustaining molecule targeting therapeutic agent, can be tested in cell or animal based assays, and the assay data may serve as the basis for clinical dosing design. The pharmaceutic compositions of the disclosure with tolerated toxicity and high efficacy are preferable.

Although the present invention and its advantages have been described in detail, it should be understood that various changes, substitutions and alterations can be made herein without departing from the spirit and scope of the invention as defined in the appended claims.

Although the present invention and its advantages have been described in detail, it should be understood that various changes, substitutions and alterations can be made herein without departing from the spirit and scope of the invention as defined in the appended claims.

### EXAMPLE 1

### Combination of Compound A and Anti-PD-1 Antibody had Better Anti-Tumor Effect

CT-26 WT cells were maintained at 37° C. in 5% CO₂ in RPMI 1640 medium (10-040-CV, Corning cellgro) supplemented with 10% FBS (10270-106, GIBCO) and used before the 10th subculture. At Day 0, 45 BALB/c female mice, 5-6 weeks old, were each subcutaneously injected with about 200.0 μL. of the CT-26 WT cell medium containing about 5×10⁵ cells.

At Day 9, 32 tumor-bearing mice were randomly allocated to four groups according to their tumor volumes, 8 mice per group. The rest were subject to euthanasia.

The four groups of animals were administered with an anti-m-PD-1 antibody (BE0146, BioXcell) in PBS (21-040-CVR, BioXcell) at 1.0 mg/mL, Compound A in saline at 4.0 mg/mL, the anti-m-PD-1 antibody in combination with Compound A, and the control vehicle (saline), respectively, at doses shown in Table 1 below.

TABLE 1
Dosing regimen

| | | | Dose | |
|---|---|---|---|---|
| Group | Test articles | N | (mg/kg) | Dosing Regimen |
| 1 | vehicle | 8 | saline | P.O., once per day |
| 2 | Anti-m-PD-1 | 8 | 10 | I.P., once every five days, three times in total |
| 3 | Compound A | 8 | 40 | P.O., once per day |
| 4 | Anti-m-PD-1 + | 8 | 10 | I.P., once every five days, three times in total |
| | Compound A | | 40 | P.O., once per day |

Mice were observed every day for their physical conditions and activities, and mice body weight and tumor volume were measured every Tuesday, Thursday and Sunday. The tumor volume (V) was calculated as (length×width²)/2.

At Day 23, mice were subject to euthanasia, from which tumors were collected, weighed and photographed. Mice body weights, tumor sizes and tumor weights were analyzed using the t-test in SPSS, and group differences were deemed statistically significant when the p-value was 0.05 or less.

Tumor growth inhibition (TGI) was calculated based on tumor weight or tumor volume. Further, Zheng-Jun Jin's Q value was calculated using the formula Q=E_{A+B}/(E_{A}+E_{B}−E_{A}×E_{B}) to assess the combined effect of the anti-PD-1 antibody and Compound A, wherein E_{A+B}, E_{A}, and E_{B} referred to the TGIs of the combination therapy, anti-PD-1 administration and Compound A administration, respectively. A Q value higher than 1.15 indicated a synergistic or additive effect.

The animals in the vehicle group looked vigorous, and no mouse was found dead in all four groups during the experiment. The average mice body weights and average tumor sizes from Day 9 to Day 23 in different groups were shown in Table 2 and 3, and the weights of individual tumors isolated from mice at Day 23 were listed in Table 4.

TABLE 2
Average mice body weights in vehicle and administration groups

| | Average body weight (g) | | | | | | | | |
|---|---|---|---|---|---|---|---|---|---|
| Group | Day 9 | Day 10 | Day 12 | Day 14 | Day 16 | Day 18 | Day 20 | Day 22 | Day 23 |
| 1 | 16.43 | 16.68 | 16.98 | 17.34 | 18.00 | 18.90 | 19.31 | 20.51 | 21.02 |
| 2 | 16.38 | 16.44 | 17.01 | 17.38 | 18.40 | 19.12 | 19.99 | 20.16 | 21.11 |
| 3 | 15.59 | 15.16 | 15.86 | 15.91 | 16.46 | 16.75 | 17.01 | 17.72 | 18.15 |
| 4 | 16.08 | 15.48 | 15.81 | 16.18 | 16.51 | 16.30 | 16.43 | 17.51 | 18.14 |

TABLE 3
Average mice tumor sizes in vehicle and administration groups

| | Average tumor size (mm³) | | | | | | | | |
|---|---|---|---|---|---|---|---|---|---|
| Group | Day 9 | Day 10 | Day 12 | Day 14 | Day 16 | Day 18 | Day 20 | Day 22 | Day 23 |
| 1 | 173.96 | 210.40 | 442.90 | 550.17 | 916.09 | 1310.74 | 1815.88 | 2296.23 | 2319.68 |
| 2 | 194.48 | 348.18 | 602.52 | 873.05 | 1226.32 | 1673.71 | 2235.65 | 2525.48 | 2698.41 |
| 3 | 176.94 | 272.49 | 492.26 | 672.51 | 876.22 | 1099.45 | 1348.89 | 1698.25 | 1870.89 |
| 4 | 191.99 | 247.43 | 412.12 | 619.61 | 762.56 | 876.64 | 1011.48 | 1020.03 | 1130.01 |

TABLE 4
Individual and group average mice tumor weights in vehicle and administration groups

| | Tumor weight (g) | | | | | | | | |
|---|---|---|---|---|---|---|---|---|---|
| | Mouse | Mouse | Mouse | Mouse | Mouse | Mouse | Mouse | Mouse | |
| Group | no. 1 | no. 2 | no. 3 | no. 4 | no. 5 | no. 6 | no. 7 | no. 8 | Average |
| 1 | 2.43 | 2.21 | 3.69 | 1.03 | 1.84 | 3.03 | 4.42 | 3.99 | 2.83 |
| 2 | 3.4 | 0.09 | 3.89 | 4.45 | 2.4 | 0.92 | 4.87 | 3.59 | 2.95 |
| 3 | 1.14 | 2.11 | 2.11 | 0.89 | 1.68 | 2.16 | 2.73 | 2.64 | 1.93 |
| 4 | 1.36 | 2.43 | 1.26 | 0.45 | 0.82 | 1.71 | 1.06 | 0.54 | 1.20 |

As shown in Table 2, the mice in each group generally had their body weights increased from Day 9 to Day 23, partly due to the tumor growth. If the tumor weight was deducted, the mice weights in the vehicle, anti-PD-1, Compound A and combination therapy groups were 18.19 g, 18.16 g, 16.22 g, and 16.94 g, respectively, at Day 23. It can be seen that the mice in Compound A group and the combination group were lighter than those in the anti-PD-1 group, suggesting that compound A might be more toxic than the anti-PD-1 antibody, and the combination therapy did not have the toxicity increased. Actually, the mice in the combination therapy group were even slightly heavier than those in Compound A group.

According to Table 3, tumor volumes kept increasing in all four groups, but the increase became slower in the Compound A and combination administration groups after Day 16. At Day 23, the tumor weights in the combination group were significantly lower than those in the anti-PD-1 or Compound A group, as shown in Table 4. Further, the tumor volume based TGIs in the Compound A, combination administration and anti-PD-1 groups at Day 23 were 19.35%, 51.29% and −16.33%, respectively, while the tumor weight based TGIs in these groups were 31.71%, 57.46% and −4.28% respectively. The Zheng-jun Jin's Q values based on tumor volume and tumor weight based TGIs were 8.2994 and 1.9961 respectively, both higher than 1.15.

The data above indicated that Compound A worked synergistically with the anti-PD-1 antibody, resulting in enhanced anti-tumor effect with equal or slightly lower toxicity.

1. A method for treating cancer in a subject in need thereof, comprising administering the subject an therapeutically effective amount of a compound of formula (I) or a pharmaceutically acceptable salt or solvent thereof, in combination with an immunotherapeutic agent or a therapeutic agent targeting a cancer-promoting/sustaining molecule, 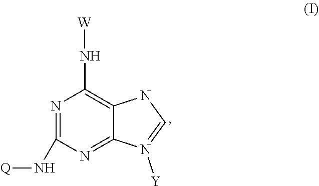
wherein W is hydrogen, an optionally substituted C₁₋₆ alkyl, an optionally substituted C₃₋₆ cycloalkyl, or an optionally substituted C₁₋₆ haloalkyl,
Y is hydrogen, or a saccharide, and
Q is hydrogen, or one selected form the group consisting of: 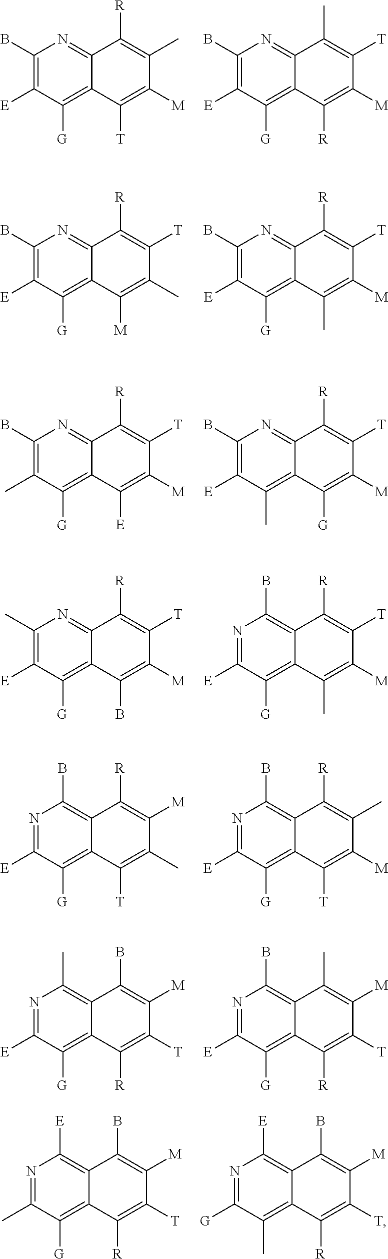
wherein B, E, G, R, T and M are independently hydrogen, an C₁₋₆ alkyl, an C₃₋₆ cycloalkyl, a halogen, a cyano, or an amino group.

2. The method according to claim 1, wherein W is selected from the group consisting of:

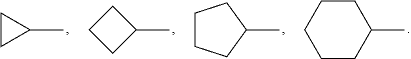

3. The method according to claim 1, wherein Q is selected from the group consisting of:

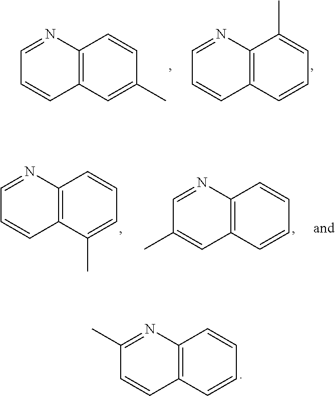

4. The method according to claim 1, wherein Y is a saccharide selected from the group consisting of: 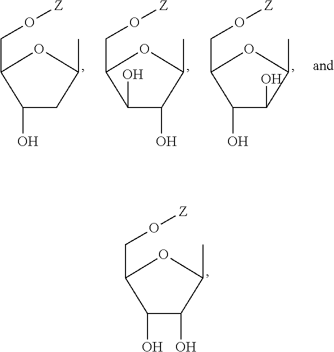
wherein Z is hydrogen or one selected from the group consisting of:

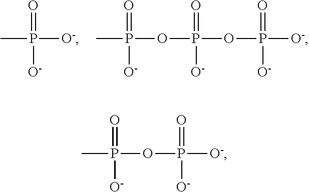

5. The method according to claim 3, wherein W is

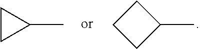

6. The method according to claim 5, wherein Q is

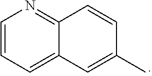

7. The method according to claim 1, wherein the compound of formula (I) is selected from the group consisting of

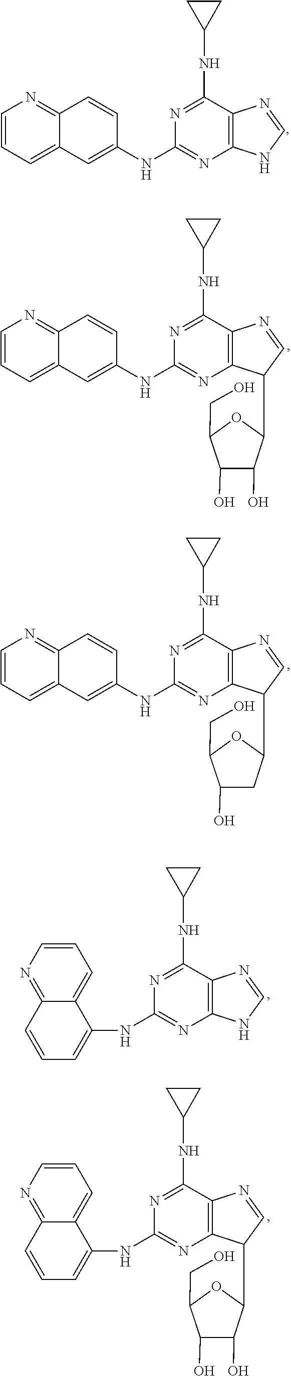

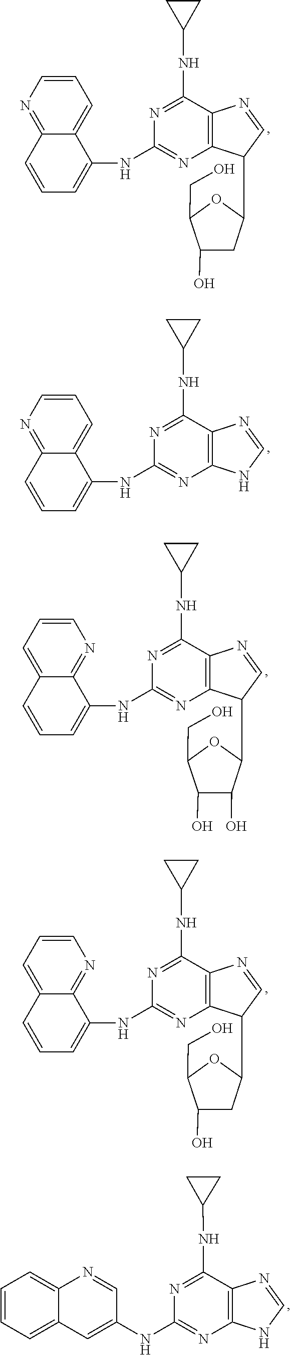

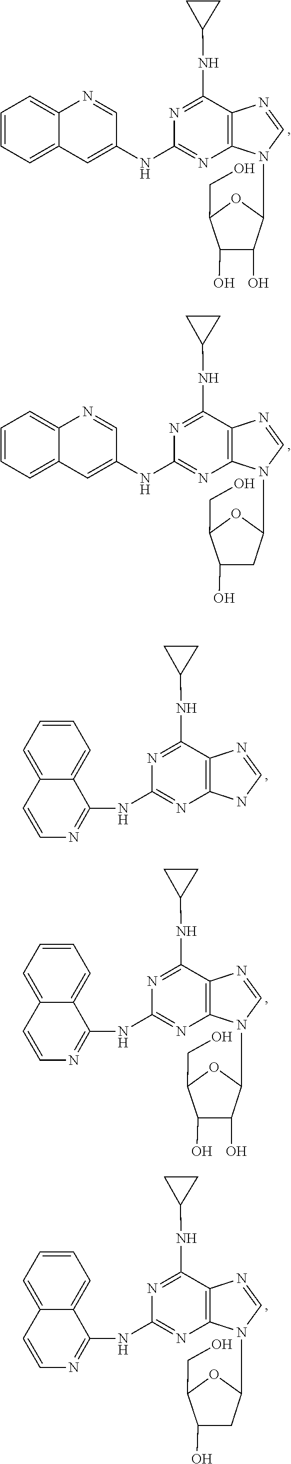

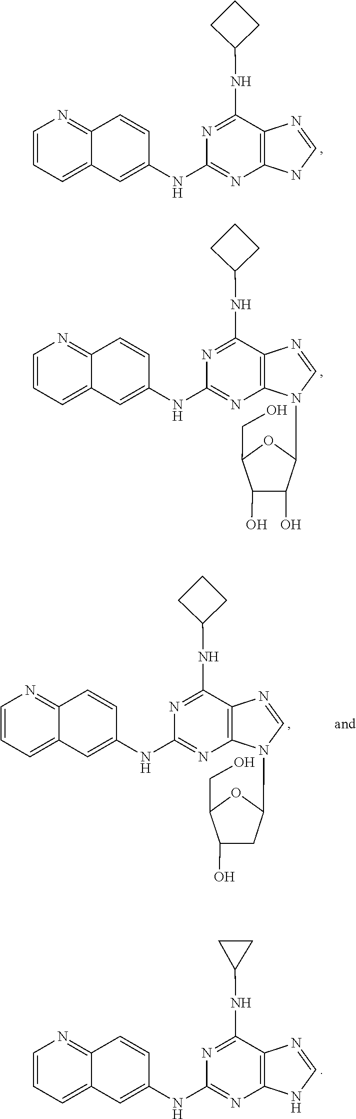

8. The method according to claim 7, wherein the compound is

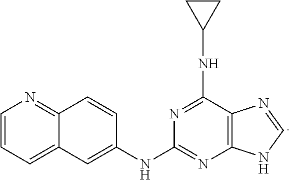 [SMILES: c1cnc2ccc(Nc3nc(NC4CC4)c4nc[nH]c4n3)cc2c1]

9. The method according to claim 1, wherein the immunotherapeutic agent or the therapeutic agent targeting a cancer-promoting/sustaining molecule is an inhibitor of PD-1, PD-L1, CTLA-4, HER-2, CD20, CD33, or CD52.

10. The method according to claim 9, wherein the immunotherapeutic agent or the therapeutic agent targeting a cancer-promoting/sustaining molecule is an antibody targeting PD-1, PD-L1, CTLA-4, HER-2, CD20, CD33, or CD52.

11. The method according to claim 9, wherein the immunotherapeutic agent or the therapeutic agent targeting a cancer-promoting/sustaining molecule is an antibody-drug conjugate targeting PD-1, PD-L1, CTLA-4, HER-2, CD20, CD33, or CD52.

12. The method according to claim 9, wherein the immunotherapeutic agent or the therapeutic agent targeting a cancer-promoting/sustaining molecule is a CAR-T cell targeting PD-1, PD-L1, CTLA-4, HER-2, CD20, CD33, or CD52.

13. The method according to claim 10, wherein the antibody targeting PD-1 is selected from the group consisting of Nivolumab and Pembrolizumab.

14. The method according to claim 10, wherein the antibody targeting PD-L1 is selected from the group consisting of Atezolizumab, Druvalumab and Avelumab.

15. The method according to claim 1, wherein the cancer is a solid cancer selected from the group consisting of lung, prostate, ovarian, brain, breast, skin, bladder, colon, gastrointestinal, head and neck, gastric, pancreas, neurologic, renal, and liver cancer.

16. The method according to claim 1, wherein the cancer is a hematological cancer selected from the group consisting of lymphocytic leukemia, myeloid leukemia, non-Hodgkin lymphoma, and Hodgkin lymphoma.

17. The method according to claim 16, wherein the myeloid leukemia is acute myeloid leukemia.
